(19) United States
(12) Patent Application Publication
(10) Pub. No.: US 2025/0366760 A1
(43) Pub. Date: Dec. 4, 2025

(54) ONE-PIECE MULTI-CONTACT MEDICAL CONNECTOR

(71) Applicant: AD-TECH MEDICAL INSTRUMENT CORPORATION, Oak Creek, WI (US)

(72) Inventor: Jonah Hinds, Milwaukee, WI (US)

(73) Assignee: AD-TECH MEDICAL INSTRUMENT CORPORATION, Oak Creek, WI (US)

(21) Appl. No.: 19/199,571

(22) Filed: May 6, 2025

Related U.S. Application Data
(60) Provisional application No. 63/652,355, filed on May 28, 2024.

(51) Int. Cl. A61B 5/293; H01R 24/58

(52) U.S. Cl. CPC A61B 5/293; H01R 24/58; A61B 2562/227; H01R 2201/12

(57) ABSTRACT

An electrical connector connects a linear-array multi-contact tail of an in-body multi-contact medical electrode device to an outgoing cable for individual electrical connection to Electroencephalography equipment. The connector uses a latch and a retention seat to secure the tail of the electrode reliably and conveniently to the connector. The retention seat holds the tail in a fixed longitudinal position in the connector.

### CROSS-REFERENCE TO RELATED APPLICATION

The present application claims priority to U.S. Provisional Patent Application No. 63/652,355, filed May 28, 2024, the content of which is incorporated herein by reference in its entirety.

### FIELD OF THE INVENTION

This invention is related generally to electrical connectors for use in the medical field and, more particularly, to medical connectors for implantable medical electrodes with multi-contact tails.

### BACKGROUND OF THE INVENTION

Multi-contact medical electrodes are placed in the human body for various purposes, such as brain-mapping in epilepsy treatment. In such treatments, a cable of wires generally extends through the medical electrode to a multi-contact tail. The multi-contact tail is linear in shape and contains an array or series of sleeve-like contacts spaced along its length. The contacts on the tail are configured to facilitate quick electrical connection of the medical electrode for monitoring, recording and analysis purposes. Various connectors have been configured to simultaneously engage the multiple contacts on electrode tails and provide individual electrical connection to separate wire strands that emerge from the connector to, for example, EEG equipment.

Examples of such prior art multi-contact medical connectors are those disclosed in the following U.S. Pat. No. 4,850,359 (Putz), U.S. Pat. No. 4,869,255 (Putz), U.S. Pat. No. 6,415,168 (Putz), U.S. Pat. No. 4,744,371 (Harris), U.S. Pat. No. 5,560,358 (Arnold et al.), U.S. Pat. No. 5,902,236 (Iversen), U.S. Pat. No. 4,516,820 (Kuzma), U.S. Pat. No. 4,712,557 (Harris), U.S. Pat. No. 4,461,304 (Kuperstein), U.S. Pat. No. 4,379,462 (Borkan et al.), U.S. Pat. No. 4,633,889 (Talalla et al.) and U.S. Pat. No. 4,676,258 (Inokuchi et al.); and U.S. Pat. No. 8,594,793 (Putz).

Medical connectors for use in patients susceptible to seizures must be secure. If a patient has a seizure, there is the chance that the electrical connections could be disrupted. Specifically, the multi-contact tails of the electrodes can become dislodged or broken by the involuntary movements that occur during a seizure. Therefore, it is important that the connector be secure so that it can withstand the jerking motions that are characteristic of seizures. If a connector does become dislodged or broken it can result in loss of information and time.

Assignee owns U.S. Pat. No. 8,594,793, entitled “Electrical Connector with a Canopy for an In-Body Multi-Contact Medical Electrode Device,” filed on Nov. 20, 2007, by David A. Putz, which is incorporated herein by reference. The connector in the '793 patent has two pivotally attached elongated members. The first elongate member includes a tail-receiving void from its proximal end to a stop at its distal end. In use, the electrode tail is inserted into the void until the tail hits the stop at the distal end of the void. The tail-receiving first elongate member also has a presentation face that is parallel to the void and notches along the length of the presentation face that intersect with the void to expose the tail contacts when the tail is fully inserted in the void. The second elongate member has a nesting surface and a series of electrical conductors (e.g. spring pin connectors) along the nesting surface. Medical staff closes the first and second elongate members with their fingers after the electrode tail is inserted into the first elongate member to make contact with the electrical connectors on the nesting surface. The connector in the '793 patent also includes a snap engagement feature that ensures the connector remains in the closed position. More specifically, one or more canopies extending from the nesting surface adjacent and partially encircling one or more pins and are configured to fit into a respective notch on the presentation face of the first, tail-receiving elongate member in order to close the connector. The canopies press against the metallic connectors and help to keep the tail secure in the elongated void. The second elongated member also includes locking tabs along its length that snap over the edge of the first elongated member, to snap the connector shut. The combination of the canopies and the tabs makes the connector somewhat difficult to close and to open. The spring pins are effective to achieve reliable contact with the contacts on the tail when the connector is closed, but it is desirable to improve the security and reliability of the connection in some instances when violent seizures or other sudden movement occurs.

It is an object of the present invention to provide a connector for medical electrodes with multi-contact tails that overcomes some of the problems and shortcomings associated with the prior art. Another object of the invention is to provide a multi-contact medical connector that is more reliably secure than the prior art given the involuntary jerking motions which are customary with a seizure condition, while at the same time enabling relatively easy opening and closing of the connector by medical staff.

These and other objects of the invention will be apparent from the following descriptions and from the drawings.

### SUMMARY OF THE INVENTION

This invention is an electrical connector for connecting a linear-array multi-contact tail of an in-body multi-contact medical electrode device. The connector uses a latch and a retention seat to reliably and conveniently secure the tail of the electrode to the connector.

The connector includes a tail-receiving elongated member pivotally attached to a base elongated member. These members are preferably made of Delrin® acetal homopolymer, which is CNC machined. The tail-receiving elongated member has a tail-receiving void that runs longitudinally through an elongated tail-receiving body. The tail of the electrode is inserted into the longitudinal tail-receiving void in the tail-receiving elongated member to use the connector. The body of the elongated tail-receiving member has notches along its length that intersect with the tail-receiving void to expose the multiple contacts of the tail installed in the tail-receiving elongated member for electrical connection. The base elongated member has an array of electrical pin conductors, e.g. spring-loaded, for the purpose of making electrical contact with the multiple electrical contacts of the tail installed in the tail-receiving elongated member when the tail-receiving elongated member and the base elongated member are pivoted together into a closed position. The tail-receiving elongated member has an opening on its proximal end leading to the tail receiving void and a stop on its distal end to facilitate alignment of the electrical contacts on the electrode tail within the notches and the spring-loaded pin conductors on the base elongated member when the tail-receiving elongated member and the base elongated member are pivoted into a closed position.

In accordance with the invention, a latch bar is provided on one of the elongated members, preferably the base elongated member, and a latch hook on the other elongated member, and a retention seat is on the proximal end of the base elongated member. The retention seat engages and retains the tail of the electrode in connector when the tail-receiving elongated member and the base elongated member are pivoted into a closed position and the latch hook is engaged over the latch bar. More specifically, the retention seat engages the soft tubing of the electrode tail and compresses the soft tubing adjacent the opening in the tail-receiving elongated member leading to the tail-receiving void and the first proximal notch.

As mentioned, the pin conductors on the base elongated member are preferably spring-loaded pins so as to maintain robust reliable contact with the respective electrical contacts on the tail. The pins are not meant or required to maintain the positioning of the tail within the connector, however. This is accomplished by the compression caused by the retention seat.

The retention seat is preferably located within the first notch on the proximal end of the tail-receiving elongated member when the connector is closed and latched shut. This first notch is wider than the other notches of the tail-receiving elongated member. The retention seat in the exemplary embodiment has a top surface with a V-shaped indentation. The compression of the soft tubing results in a gap in the tail receiving void which is smaller than the metal contacts, therefore disallowing the electrode to become dislodged from the connector. This configuration enables the connector to provide a robust mechanical connection of the tail.

The latch on the connector is also configured to facilitate convenient use, and a robust and reliable closed position. The latch is located on the proximal end of the connector, which provides force directly to the retention seat when the latch is closed and is therefore less susceptible to bending or warping. The tail-receiving elongated member has a handle forming part of the latch hook, which provides leverage and a convenient place to squeeze close or pull open the latch. The base elongated member also has a handle, preferably two laterally spaced handles, which are accessible to the sides of the handle on the tail-receiving elongated member when the latch is closed. The handles also provide leverage and a convenient place to squeeze close or pull open the latch. In addition, the configuration of the latch hook in relation to the latch bar facilitates reliable robust locking of the latch, and convenient closing and opening of the latch. In this regard, the centerline of the latch bar is preferably offset slightly (0.02 inches) below the pivot axis of the tail-receiving elongated member and the base elongated member. This offset helps to maintain the latch closed unless it is intended to be opened, without making the latch hook fit over the latch bar too tightly. Further, the leading edge of the latch hook is at a downward angle of approximately 27° when the latch hook is closing and initially engages the latch bar. And, the contacting edges of the latch hook are rounded. The combination of these features enables a snap over latch fitting without requiring too much force to be convenient for attending physicians, nurses, or technicians.

Other features of the invention may be apparent to those skilled in the art upon reviewing the following drawings and description thereof.

### BRIEF DESCRIPTION OF THE DRAWINGS

The drawings illustrate a preferred embodiment including the above-noted characteristics and features of the invention. The invention will be readily understood from the descriptions and drawings. In the drawings:

FIG. 1 is a perspective view of a connector, constructed in accordance with an exemplary embodiment of the invention, with the plural-contact tail of an in-body medical electrode in position for insertion into the connector.

FIG. 2 is a perspective view of the connector of FIG. 1, showing the electrode tail fully inserted into the connector with the connector in an open position.

FIG. 3 is a perspective view of the connector of FIG. 1, showing the electrode tail fully inserted into the connector and the connector in a closed position.

FIG. 4 is a partially sectional view taken along line 4-4 in FIG. 3, which illustrates certain internal details of the mechanical and electrical connections between the connector and the plural-contact tail of the electrode.

FIG. 5 is a sectional view taken along line 5-5 in FIG. 4, showing in particular the connector in a closed position and the connector squeezing the soft tubing of the plural-contact tail of the electrode.

FIG. 6 is a sectional view taken along line 6-6 in FIG. 4, showing in particular the connector in a closed position and the connector latch in a closed position and showing one of the metal electrical contacts of the tail making electrical contact with a spring-loaded pin.

### DETAILED DESCRIPTION OF AN EXEMPLARY EMBODIMENT

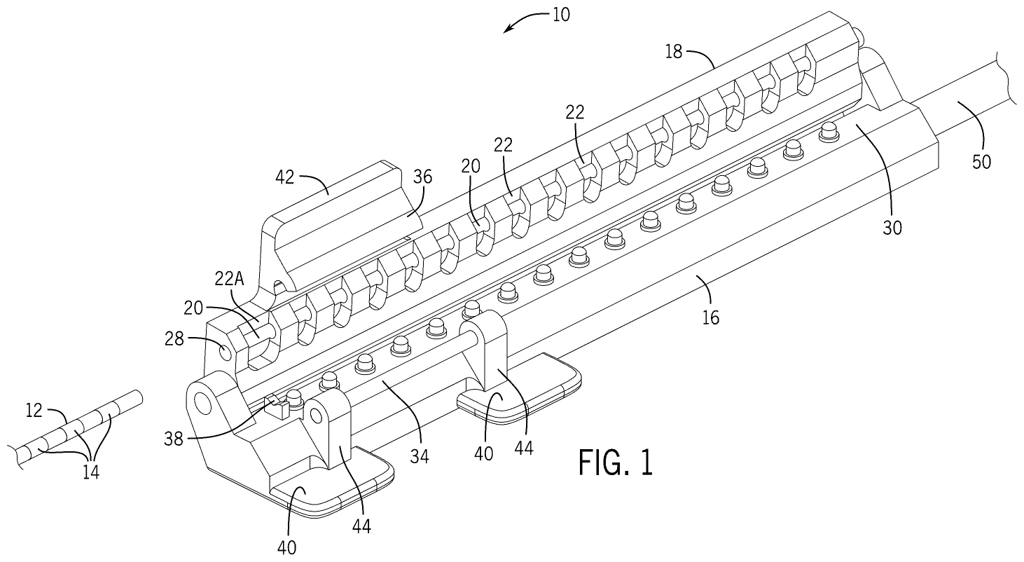

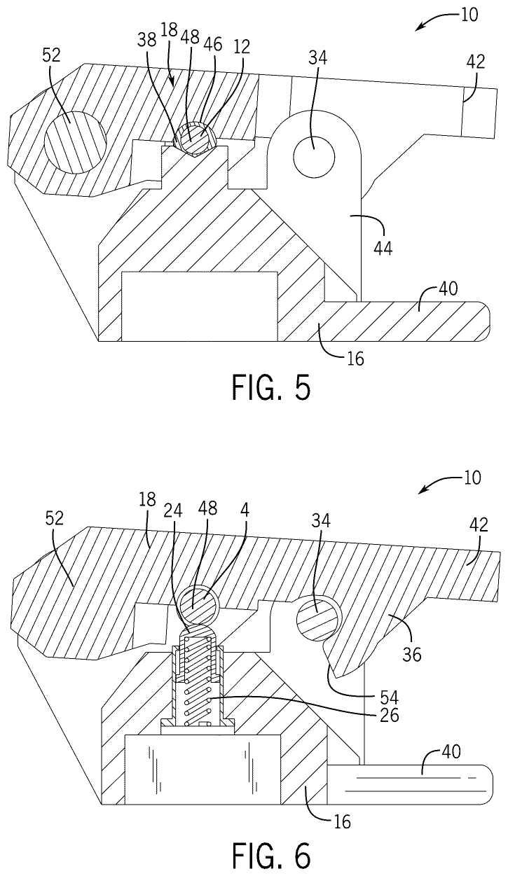

FIGS. 1-6 illustrate an electrical connector 10 for connecting the linear-array, plural-contact tail 12 of an in-body multi-contact medical electrode (the in-body portion of which is not shown), having a linear array of electrical contacts 14 spaced along its length. The electrical contacts 14 are each electrically linked by a small diameter electrical wire running up and beyond tail 12 to a particular in-body contact on the in-body portion of the electrode, as known in the prior art. Soft tubing 46 contains the multiple internal electrode contact wires, and also spans between the electrical contacts 14 on the tail 12 of electrode. The connector 10 includes a base elongated member 16 and a tail-receiving elongated member 18 which are pivotable with respect to one another about a pivot axis 52 (see FIG. 5) which extends along their lengths. The bodies of the base elongated member 16 and a tail-receiving elongated member 18 are desirably made from Delrin® acetal homopolymer (Polyoxymethylene POM), which is CNC machined. This material has suitable strength for repeated use and provides low-friction and high-wear resistance. These characteristics are desirable since the connector 10 is meant to be re-usable (e.g., rated life of at least 5 uses) and it is important that it maintains reliability and ease of use throughout its rated life, and also that it can withstand sterilization processes.

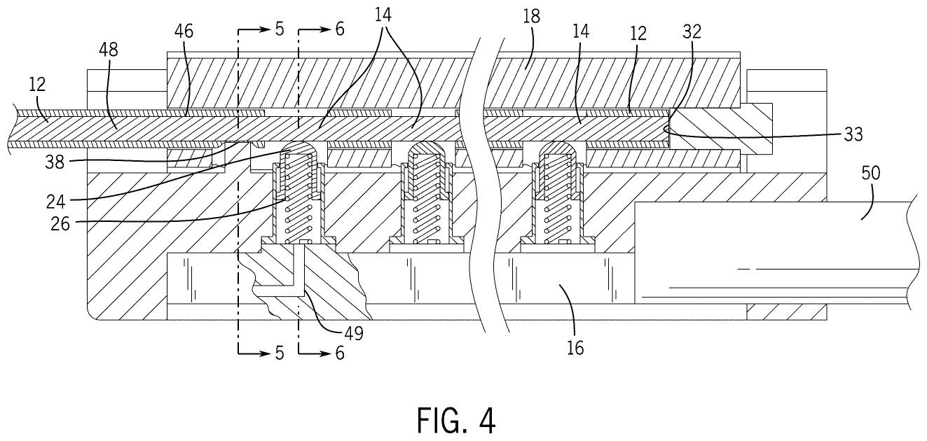

The tail-receiving elongate member 18 extends along the length of base elongate member 16 and includes a linear tail-receiving void 20, and notches 22 along its length. The notches 22 intersect with the linear void 20 to expose contacts 14 of tail 12 in alignment with a respective intersecting pinhead 24 on the base elongate member 16. The pinheads 24 on the elongate base member 16 are each preferably gold-plated, part of a spring-loaded pin plunger device 26 as depicted in FIGS. 4 and 6. The base elongate member 16 has nesting surface 30 for a linear array of the spring-loaded pin plunger devices 26, which are in alignment with the notches 22 on the tail-receiving elongate member 18. The connector 10 in the embodiment shown in FIG. 1 includes sixteen of the spring-loaded pin plunger devices 26 and is designed to connect the tail 12 of an electrode having sixteen (16) or less contact 14. In most circumstances, the connector 10 having sixteen (16) spring-loaded pin plunger devices 26 will be used to connect to a tail 12 having sixteen (16) contacts 14.

The tail-receiving, elongated member 18 has an opening 28 on its proximal end leading to the elongated void 20. The tail 12 is inserted through the opening 28 into the elongated void 20 until the contacts 14 on the tail 12 align with the notches 22.

A stop 32 (FIG. 4) is provided at the distal end of the elongated void 20, which abuts the leading end 33 of the tail and facilitates alignment of the electrical contacts 14 on the electrode tail 12 within the notches 22 on the tail-receiving elongate member 18. If the tail 12 has less than sixteen (16) electrical contacts 14, the stop 32 is not used and the tail 12 is partially inserted and aligned by sight for the retention feature to operate properly.

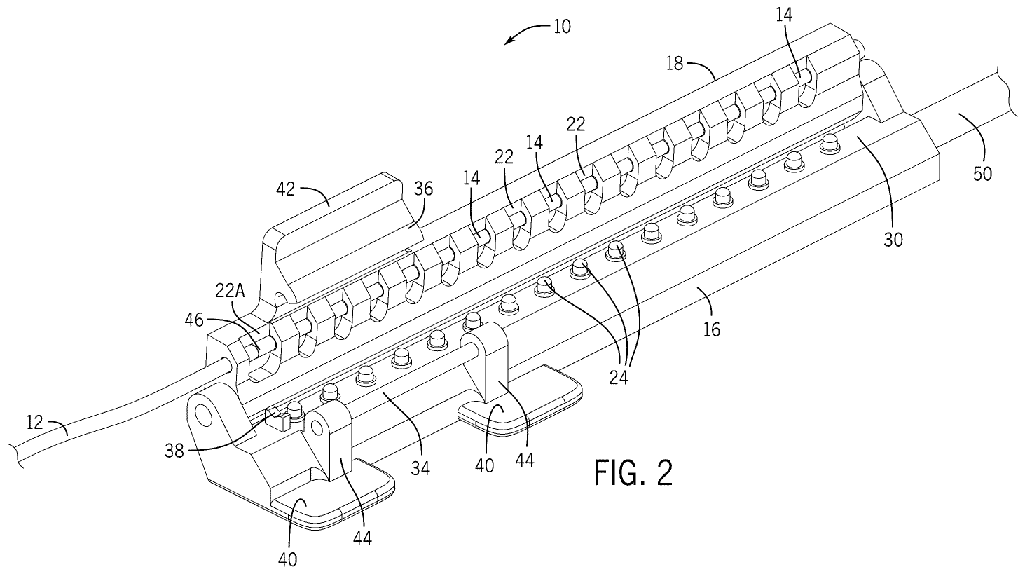

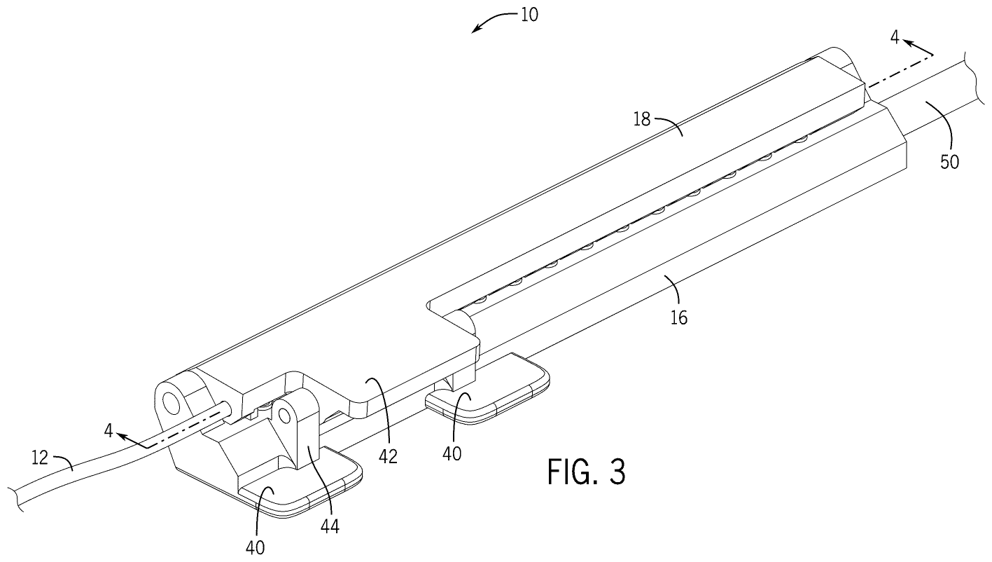

A latch bar 34 is mounted on the base elongated member 16, preferably near the proximal end of connector 10 into which the tail 12 is inserted. A latch hook 36 is integral with the tail-receiving elongated member 18 and is positioned to snap engage over the latch bar 34 when the tail-receiving elongated member 18 is pivoted relative to the base elongated member 16 into a closed position, see FIG. 3. The base elongated member 16 also includes a retention seat 38 on its proximal end. The first notch 22A on the proximal end of the tail-receiving elongated member 18 is wider than the other notches 22 so that it provides clearance for the retention seat 38 when closing the tail-receiving elongated member 18, see e.g. FIG. 2. The retention seat 38 engages and retains the tail 12 of the electrode in connector 10 when the tail-receiving, elongated member 18 and the base, elongated member 16 are engaged in the closed position and the latch hook 36 is engaged over the latch bar 34. The retention seat 38 shown in the drawings has a V-shaped top surface for engaging and supporting the tail 12. Referring to FIG. 3, the latch hook 36 covers the pin connectors 26 on the proximal end from view. In the exemplary embodiment shown, there is a longitudinal space on the distal end of connector 10 between the base elongated member 16 and tail-receiving elongated member 18 when the connector 10 is closed. If desired, the base elongated member 16 can be made with a longitudinal wall to cosmetically cover this space.

The base elongated member 16 has finger handles 40 that help to open and close the connector 10. Similarly, the latch hook 36 on the tail-receiving elongated member 18 includes a finger handle 42 for the same purpose. There is space between the handles 40 on the base, elongated member 16 to facilitate access to the handle 42 on the tail receiving elongated member 18. The base elongated member 16 also includes upstanding support legs 44 for the latch bar 34. The latch bar 34 is desirably a stainless-steel rod that is press fit into holes through the support legs 44.

As mentioned, the tail 12 of the electrode has soft tubing 46 surrounding the multiple conductors/wires. When the tail 12 is fully inserted into the elongated void 20 as shown for example in FIG. 2, a portion of the soft tubing 46 is aligned with the retention seat 38. When the connector 10 is closed as in FIGS. 3 and 4, the portion of soft tubing 46 aligned with the retention seat 38 is compressed to hold the tail 12 longitudinally in place within the connector 10. FIG. 5 shows a close-up cross-sectional view of the compression of the soft tubing 46 by the retention seat 38. The soft tubing 46 on the tail 12 of the electrode has contacts 14 with the same diameter as the soft tubing 46, so when the soft tubing 46 is compressed by the retention seat 38 the tail 12 is unable to exit the tail receiving void 28.

FIGS. 4 through 6 depict the internal wiring 48 of the tail 12 in a general sense. It should be understood that the internal wiring 48 in fact contains multiple isolated conducting wires (16 in the exemplary embodiment), with each isolated conducting wire being connected to a respective contact 14 on the tail 12. Similarly, each spring-loaded pin connector 26 is connected to an isolated electrical conducting wire 49 (FIG. 4) in the base elongated member 16. The electrical conductors in the base elongated member 16 exit the base elongated member 16 as a group in a cable 50.

Referring to FIGS. 5 and 6, it is important that the latch hook 36 closed reliably over the latch bar 34 so that the retention seat 38 maintains a suitable amount of holding pressure on the soft tubing 46 of the tail 12 when the connector 10 is closed. With the tail 12 being held in place longitudinally by the retention seat 38, the tension of the spring-loaded pin connectors 26 is not needed to hold the tail 12 in a fixed longitudinal position. While it is important that the latch hook 36 closed reliably over the latch bar 34, it is also important that closing the latch be convenient and that opening the latch when desired to do so is also convenient. This is accomplished by the latch configuration shown in FIGS. 5 and 6. First, the centerline of the horizontal latch bar 34 is located on a plane slightly lower (e.g. 0.02 inches lower) than the pivot point 52 of the tail-receiving elongated member 18. While the offset is not necessary, the offset helps to keep the latch reliably closed without having increase the amount of force to close the latch. Second, the leading edge 54 of the latch hook 34 makes contact with the latch bar 34 at a downward angle of approximately 27°. Third, the leading corner of the latch hook 34 is rounded, not sharp. And fourth, the handle 42 on the tail-receiving elongated member 18 and the handles 40 on the base elongated member 16 provide leverage and accessible grips to open and close the latch with one or two hands.

While the principles of this invention have been described in connection with specific embodiments, it should be understood clearly that these descriptions are made only by way of example and are not intended to limit the scope of the invention.

The invention claimed is:

1. An electrical connector for connecting a linear-array multi-contact tail of an in-body multi-contact medical electrode device, said connector comprising:
a tail-receiving elongated member pivotally attached to a base, elongated member;
the tail-receiving elongated member having a tail-receiving void that runs longitudinally through an elongated tail-receiving body, the elongated tail-receiving body having notches along its length that intersect with the tail-receiving void to expose the multiple contacts of a multi-contact tail installed in the tail-receiving elongated member for electrical connection;
the base elongated member having a nesting surface along its length and an array of electrical pin conductors on the nesting surface for the purpose of making electrical contact with the multiple electrical contacts of a multi-contact tail installed in the tail-receiving elongated member when the tail-receiving elongated member and the base elongated member are pivoted into a closed position;
the tail-receiving elongated member having an opening on its proximal end leading to the tail receiving void and a stop on its distal end to facilitate alignment of the electrical contacts on the electrode tail within the notches on the tail-receiving elongated member and the pin conductors on the base elongated member when the tail-receiving elongated member and the base elongated member are pivoted into a closed position; and
said electrical connector further comprises a latch bar on one of the elongated members and a latch hook on the other elongated member, and a retention seat on the proximal end of the base elongated member which engages and retains the tail of the electrode in connector when the tail-receiving elongated member and the base elongated member are pivoted into a closed position and the latch hook is engaged over the latch bar.

2. The multi-contact medical connector of claim 1 wherein the pins are spring loaded pins.

3. The multi-contact medical connector of claim 1 wherein the retention seat is located such that it is located within the first notch on the proximal end of the tail-receiving elongated member when the connector is closed and latched shut.

4. The multi-contact medical connector of claim 3 wherein the first notch is wider than the other notches on the proximal end of the tail-receiving elongated member.

5. The multi-contact medical connector of claim 1 wherein the centerline of the latch bar is offset below the pivot axis of the tail-receiving elongated member and the base elongated member.

6. The multi-contact medical connector of claim 5 wherein a leading edge of the latch hook is at a downward angle of approximately 27° when the latch hook is closing and initially engages the latch bar.

7. The multi-contact medical connector of claim 1 wherein the tail-receiving elongated member includes a handle on its proximal end to facilitate opening and closing of the latch hook over the latch bar.

8. The multi-contact medical connector of claim 1 wherein the base elongated member includes a handle on its proximal end to facilitate opening and closing of the latch hook over the latch bar.

9. The multi-contact medical connector of claim 7 wherein the base elongated member includes two handles on its proximal end to facilitate opening and closing of the latch hook over the latch bar.

10. The multi-contact medical connector of claim 1 wherein the retention seat has a top surface with a V-shaped indentation.

11. The multi-contact medical connector of claim 1 wherein the electrode tail has soft tubing and the electrical contacts are made of metal, and the retention seat compresses the soft tubing when the connector is closed to a diameter less than the diameter of the metal electrical contacts.

12. The multi-contact medical connector of claim 11 wherein the outside diameter of the metal electrical contacts and the uncompressed soft tail is the approximately the same.
